(19) United States
(12) Patent Application Publication
(10) Pub. No.: US 2019/0250127 A1
(43) Pub. Date: Aug. 15, 2019

(54) TRANSLOCATION OF A NON-NUCLEIC ACID POLYMER USING A POLYMERASE

(71) Applicant: ARIZONA BOARD OF REGENTS on behalf of ARIZONA STATE UNIVERSITY, Scottsdale, AZ (US)

(72) Inventors: Peiming ZHANG, Gilbert, AZ (US); Stuart LINDSAY, Phoenix, AZ (US)

(21) Appl. No.: 16/336,871

(22) PCT Filed: Sep. 26, 2017
(86) PCT No.: PCT/US2017/053561

§ 371 Date: Mar. 26, 2019

Related U.S. Application Data
(60) Provisional application No. 62/400,530, filed on Sep. 27, 2016.

(51) Int. Cl. G01N 27/447; G01N 33/487

(52) U.S. Cl. CPC G01N 27/44791; G01N 33/48721

(57) ABSTRACT

Apparatus and means by which a polysaccharide or other heterogeneous polymer is concatenated with a nucleic acid polymer that is captured by a primer on a polymerase tethered to a bead trapped by a nanopore. The translocation of the nanopore by the polysaccharide or other heterogeneous polymer is then controlled by the speed at which the polymerase releases newly synthesized nucleic acid or slows the motion of the nucleic acid as it is pulled on by an electrophoretic field.

### RELATED APPLICATIONS

This application claims priority to and the benefit of U.S. Ser. No. 62/400,530, filed Sep. 27, 2016, the contents of which are incorporated herein by reference in their entireties.

### GOVERNMENT SUPPORT

This invention was made with government support under R01 HG006323 awarded by the National Institutes of Health. The government has certain rights in the invention.

### FIELD

Embodiments of the present disclosure are directed to systems, methods, devices, and compositions of matter for sequencing molecules. More specifically, the present disclosure includes embodiments where a polysaccharide or other heterogeneous polymer concatenated with a nucleic acid polymer is captured by a primer on a polymerase tethered to a bead trapped by a nanopore, where the polymer may be sequenced/identified.

### BACKGROUND

Carbohydrates, particularly those glycosylating proteins and lipids (glycans), play an essential role in biological processes at all levels, such as protein folding, cell adhesion, signal transduction, pathogen recognition, and immune responses. On the other hand, the aberrant glycosylation of proteins is associated with oncogenic transformation. Over 50% of all human proteins are glycosylated. A glycome—a complete collection of glycans and glycoconjugates in a cell or organism—is diverse (e.g. 1.92×10¹¹ possible hexasaccharides formed mainly from ten of the most abundant mammalian monosaccharides) and dynamic (i.e., variation of glycoforms of proteins at different developmental stages of a cell).

Currently, mass spectrometry is the most powerful analytical technique for structural glycomics. Since many carbohydrates are epimers, anomers, and regioisomers, mass spectrometry is unable to identify those sharing a molecular weight without additional chemical steps. The problem has been addressed by combining ion-mobility spectrometry, which uses collision cross-sections to separate isomers, with mass spectrometry (IM-MS), but IM-MS cannot resolve closely related epimers because they have almost identical collision cross-sections.

Emerging nanotechnologies (e.g., nanopores for analyzing oligosaccharides) offer a promising alternative for glycomics. In US20150144506, herein incorporated by reference, an electron tunneling technique is introduced which is configured to, among other things, identify carbohydrates electronically at a single-molecule level. Some of the disclosed embodiments may be capable of analyzing nanomolar (nM) concentrations in volumes of a few microliters, using less than a picomole of sample. In some embodiments, the number of individual molecules in each subset in a population of coexisting isomers are counted, and can be quantitative over more than four orders of magnitude of concentration. For example, in some embodiments, it can resolve epimers that are not well separated by ion-mobility, and can detect glycosylation of a peptide.

Recently, we have shown that some embodiments can identify common biological mono- and di-saccharides (see, e.g., Electronic Single Molecule Identification of Carbohydrate Isomers by Recognition Tunneling, arxiv.org/abs/1601.04221), herein incorporated by reference. However, the method may only identify one molecular species at a time, so solving the combinatorially complex problem of reading the sequence of sugars in a linear polymer is very challenging.

Oligosaccharide molecules, such as glycosaminoglycans, are generally charged, and thus, can be pulled through a nanopore using an electric field. However, they are very small, requiring a very small (one nanometer diameter) nanopore to ensure that each sugar residue passes the reading element in turn. Their small size also means that they move very rapidly in an electric field because they present a small friction to the surrounding water. Thus, even if they could be passed through a constriction small enough to ensure that only one sugar residue at a time lies in the reading region of the device, they would spend too little time in the reading region to generate a signal that could be read. This is because tunneling signals are typically picoamps, so millisecond data acquisition times are needed for typical device capacitances of a few pF.

The same problem has been addressed in the case of DNA sequencing, using a DNA polymerase to both clamp the DNA and to regulate the speed with which it can be pulled through a nanopore. However, currently, no equivalent of a DNA polymerase is known to exist for oligosaccharides.

### SUMMARY OF SOME OF THE EMBODIMENTS

Some embodiments of the current disclosure introduce a device that uses a DNA polymerase to regulate the motion of an oligosaccharide, as well as to hold it in place so that it can be captured in a reading junction embedded in a pore that is much larger than the diameter of the sugar molecule. Such embodiments, enables the use of larger pores to identify oligosaccharides and the like, addressing the difficulty in manufacturing small (nm-diameter) pores.

Some of the disclosed embodiments may be use in association with the embodiment disclosed in (especially disclosed molecule sequencing/identification system embodiments, and in some cases, the system recited in claim 1), of U.S. Pat. No. 9,395,352 (Lindsay et al.), herein incorporated by reference in its entirety.

In some embodiments, an apparatus for sequencing a heteropolymer is provided and may include: (a) a substrate, (b) a pair of electrodes proximate to or within the constriction and separated by a gap of between 0.5 to 10 nm, (c) a constriction arranged within the substrate and configured with a size and operatively arranged with the gap such that a heteropolymer molecule to be sequenced passes through the constriction, (d) means for reading an electrical signal characteristic of the molecule from the pair of electrodes as the heteropolymer molecule passes through the constriction and becomes electrically connected with the electrodes, (e) a bead having a size that is greater than a size of the constriction, (f) a DNA-binding protein attached to the bead, and (g) a DNA polymer bound to the DNA-binding protein and configured to bind with a heteropolymer for sequencing by the apparatus. In some embodiments, the heteropolymer is not a nucleic acid.

The above noted embodiments are further clarified, and/or may further include one and/or another of the following feature(s)/functionality(ies):
the bead is sized such that it cannot move into the constriction;
the heteropolymer includes an oligosaccharide;
the heteropolymer includes a peptide;
the heteropolymer includes a protein;
the heteropolymer includes a glycoprotein;
the heteropolymer is tethered to a charged polymer;
tethering of the charged polymer is such that it is drawn into the constriction;

In some embodiments, a method for preparing a heteropolymer for sequencing is provided and may include attaching a DNA-binding protein to a bead, the bead having a size greater than a size of a constriction of a sequencing apparatus, binding a DNA polymer to the DNA-binding protein, and binding a heteropolymer to the DNA polymer.

In some embodiments, a method for sequencing a heteropolymer in a sequencing apparatus having a constriction is provided and may include: (a) attaching a DNA-binding protein to a bead, the bead including a size greater than a size of a constriction of a sequencing apparatus, the sequencing apparatus further including a substrate, the constriction arranged within the substrate and configured with a size and operatively arranged with a pair of electrodes separated by a gap of between 0.5 to 10 nm such that a heteropolymer molecule to be sequenced passes through the constriction, reading means for reading an electrical signal characteristic of a heteropolymer molecule being sequenced from the pair of electrodes as the molecule being sequenced becomes electrically connected to the electrodes; (b) binding a DNA polymer to the DNA-binding protein; (c) binding a heteropolymer for sequencing to the DNA polymer; (d) arranging the bead to a first side of the constriction; and (e) sequencing the heteropolymer by reading the electrical signals thereof as the heteropolymer passes through the constriction.

In some embodiments, the present disclosure also provides a method for regulating the speed of a heteropolymer passing through a constriction in a sequencing apparatus. The method comprises: (a) attaching a DNA-binding protein to a bead, the bead including a size greater than a size of a constriction of a sequencing apparatus; (b) binding a DNA polymer to the DNA-binding protein; (c) binding a heteropolymer for sequencing by the sequencing apparatus to the DNA polymer; (d) arranging the bead to a first side of the constriction of the sequencing apparatus, wherein the first side of the constriction is in fluid communication with a reservoir having free nucleotides; and (e) regulating a speed of the heteropolymer for sequencing through the constriction by varying a concentration of the free nucleotides in the reservoir. In some embodiments, the concentration of the free nucleotides is increased such that the heteropolymer for sequencing increases speed through the constriction.

### BRIEF DESCRIPTION OF THE FIGURES

FIG. 1: Control of DNA translocation through a nanopore according to the prior art.

FIG. 2: Means for fixing the location of a polymer with respect to the electrodes in a recognition tunneling junction according to some embodiments.

FIGS. 3A-3B: Comparison of recognition tunneling signals obtained as free DNA oligomers pass the recognition tunneling junction (FIG. 3A) and as an oligomer fixed as in FIG. 2 interacts with the recognition tunneling junction (FIG. 3B), according to some embodiments.

FIG. 4: Apparatus for controlling the translocation of a non-DNA polymer by coupling it to DNA bound with a DNA polymerase according to some embodiments.

FIG. 5: Scheme for coupling a non-DNA polymer with a DNA hairpin for forward and reverse translocation control according to some embodiments.

FIG. 6: Coupling of the polymerase-DNA complex to a bead used to fix its location with respect to a recognition tunneling junction according to some embodiments.

FIG. 7: Rolling-circle amplification method for controlling translocation of a non-DNA polymer according to some embodiments.

FIG. 8: Scheme for coupling DNA to the terminal lactose of a glycan according to some embodiments.

FIG. 9: Detail of the oxime coupling reaction according to some embodiments.

### DESCRIPTION OF SOME OF THE EMBODIMENTS OF THE DISCLOSURE

### Definitions

It is to be understood that the terminology used herein is for the purpose of describing particular embodiments only and is not intended to be limiting. It must be noted that, as used in the specification and the appended claims, the singular forms “a,” “an” and “the” include plural referents unless the context clearly dictates otherwise.

The term “and/or” is used in this disclosure to mean either “and” or “or” unless indicated otherwise.

As used herein, the term “heteropolymer” refers to a polymer having at least two monomer units, and where at least one monomeric unit differs from the other monomeric units in the polymer. In some embodiments, the heteropolymer is the molecule to be sequenced.

As used herein, the term “peptide” refers to a short polypeptide, e.g., one that typically contains less than about 50 amino acids and more typically less than about 30 amino acids. The term as used herein encompasses analogs and mimetics that mimic structural and thus biological function.

As used herein, the term “bead” can include any object. The bead can be in any shape or form. For example, the bead can be a sphere, a cube, a rod, a star, or any irregular shape.

The term “comprising” as used herein is synonymous with “including” or “containing”, and is inclusive or open-ended and does not exclude additional, unrecited members, elements or method steps. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

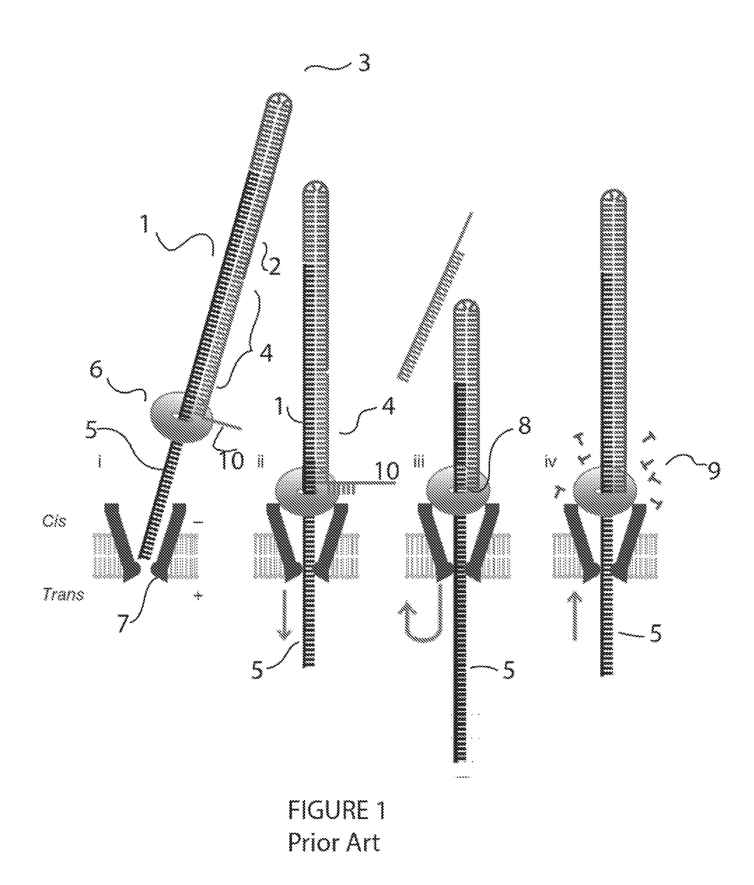

Prior DNA translocation control is shown in FIG. 1 (Manrao, Derrington et al. 2012). Referring to part i of FIG. 1, the DNA to be sequenced (1) is attached to a single-stranded DNA (5) at its 5′ end and hybridized to a complementary strand (2) which is also attached to a hairpin adaptor (3). The 3′ end of the complementary strand (2) is followed by a hybridized complementary sequence containing a 3′ tail that is abasic for about 10 nucleotide repeats. This construct is loaded onto a DNA polymerase (6) which is located at the double-strand-single strand junction, a point that would normally act as a primer for the polymerase, but which is blocked in this case by the abasic part (10) of the strand (4).

The single stranded tail (5) is pulled into a nanopore (7) using an electric field. In this case, the pore is a protein pore small enough in diameter to only pass a single-stranded region. Referring to ii in FIG. 1, the force generated by the electric field in the pore on the single stranded oligomer (5) unwinds the double stranded region (1 and 4), generating an ionic current signal variation from which the sequence can be deduced.

Referring to iii in FIG. 1, once the strand (4) is displaced, a normal primer sequence becomes available (8). Referring to iv in FIG. 1, if free nucleotides (9) are present, the consequent strand synthesis pulls the single stranded region (5) back up the pore, yielding a second sequence read of the same strand in the opposite direction. In the case of this reverse read, the speed of translocation is controlled by the polymerization rate, which is itself controlled by the concentration of nucleotides.

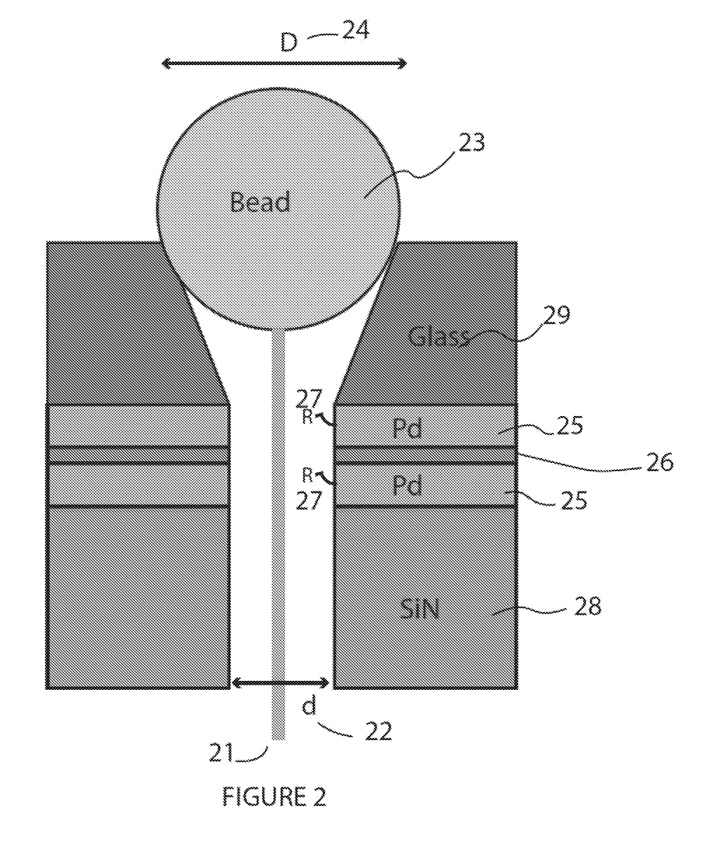

In prior disclosures, we have described a device for reading the identity of individual molecules based on recognition tunneling (e.g., see US20100084276 hereby incorporated by reference). Referring to FIG. 2, two palladium electrodes (25) are separated by a thin dielectric layer (26) such that when a channel or pore (22) of diameter d is cut through the layers, the exposed metal surfaces in the channel form a junction through which electrons can tunnel via any molecules that span the gap. In particular, the exposed surfaces of the electrodes are functionalized with reader molecules (“R”, 27) that are covalently attached to the electrodes and form weak, non-covalent bonds with the molecules to be sequenced (e.g., hydrogen bonds with the bases in a DNA chain). The nanopore in this case is a hole drilled through the electrode stack including any supporting layer (28) and any covering layer (29). It has been challenging to make pores of atomic dimensions in such complicated stacks of materials, and, moreover, small openings do not wet and are not readily amenable to chemical treatments. It is at least for these reasons that solid state nanopores have not yet replaced the protein channels currently used for DNA sequencing.

However, in an unexpected development, we have found that DNA molecules are readily trapped by the recognition molecules (27) even if the diameter of the opening (d) is much greater than the diameter of the DNA. For example, signals have been obtained with openings as big as 40 nm with single stranded DNA of diameter less than 2 nm. Thus, any fluctuation that causes the molecule to be read to become bonded to the recognition molecules (27) tends to hold the polymer chain against the wall as it passes through the pore.

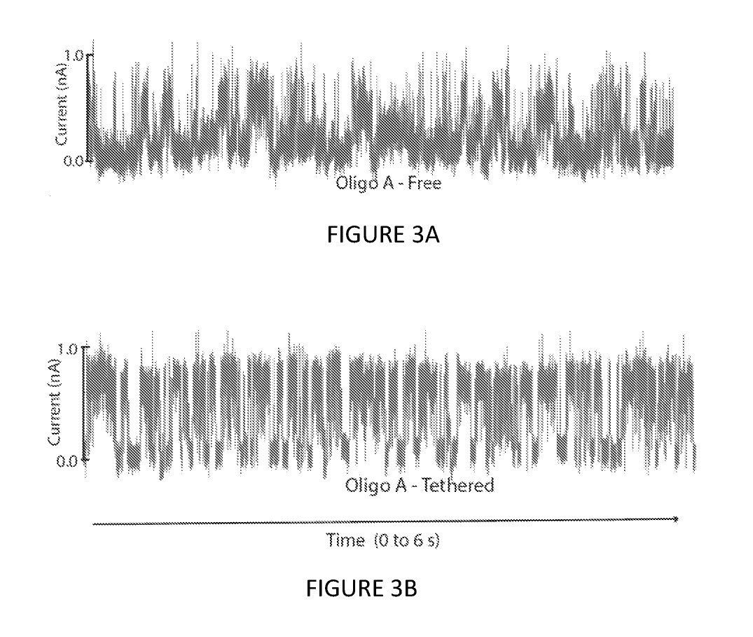

In FIG. 2, the molecule to be read (21) is shown attached to a bead (23) of diameter (24) D (>d) holding the polymer in the center of the pore. Nonetheless, signals are readily generated. FIG. 3a shows a train of signals obtained as 50 nt oligomers pass through a 20 nm diameter pore freely. The signal amplitude varies substantially, which is not surprising in view of the fact that many molecules (of <2 nm diameter) could occupy the pore (20 nm diameter) simultaneously. In the case where the polymer is tethered, the bead is functionalized with at most 2 sites that can bind a biotinylated DNA molecule, so the most probable number of molecules held in the pore is one. The result is a remarkably uniform train of signals (FIG. 3b) as the bases bind and unbind the recognition molecules. The result is very reproducible, showing that the strand is always captured by the recognition molecules. Thus, recognition tunneling, in conjunction with the use of a bead or similar method of holding the polymer over the pore will result in reads of composition of a single molecule, even if the pore is much larger than the diameter of the molecule to be sequenced. One method for achieving this clamping action is disclosed in US20160194698. In that disclosure, we described a method for attaching a molecular clamp to one of the electrodes. The attachment method for such a clamp can be by means of a bead that is physically jammed against the pore as shown in FIG. 2.

In one aspect, the present disclosure relates to an apparatus for sequencing a heteropolymer. The apparatus can include: (a) a substrate, (b) a pair of electrodes proximate to or within the constriction and separated by a gap of between 0.5 to 10 nm, (c) a constriction arranged within the substrate and configured with a size and operatively arranged with the gap such that a heteropolymer molecule to be sequenced passes through the constriction, (d) means for reading an electrical signal characteristic of the molecule from the pair of electrodes as the heteropolymer molecule passes through the constriction and becomes electrically connected with the electrodes, (e) a bead having a size that is greater than a size of the constriction, (f) a DNA-binding protein attached to the bead, and (g) a DNA polymer bound to the DNA-binding protein and configured to bind with a heteropolymer for sequencing by the apparatus. In some embodiments, the heteropolymer is not a nucleic acid. In some embodiments, the heteropolymer is selected from the group consisting of an oligosaccharide, a polysaccharide, a peptide, a protein, and a glycoprotein. The heteropolymer can be either charged or uncharged. In some embodiments, the DNA-binding protein is a DNA polymerase. The means for reading an electrical signal can be any electronic device capable of reading an electrical signal.

In another aspect, the present disclosure relates to a method for preparing a heteropolymer for sequencing. The method can include attaching a DNA-binding protein to a bead, the bead having a size greater than a size of a constriction of a sequencing apparatus, binding a DNA polymer to the DNA-binding protein, and binding a heteropolymer to the DNA polymer.

In another aspect, the present disclosure relates to a method for sequencing a heteropolymer in a sequencing apparatus having a constriction. The method can include: (a) attaching a DNA-binding protein to a bead, the bead including a size greater than a size of a constriction of a sequencing apparatus, the sequencing apparatus further including a substrate, the constriction arranged within the substrate and configured with a size and operatively arranged with a pair of electrodes separated by a gap of between 0.5 to 10 nm such that a heteropolymer molecule to be sequenced passes through the constriction, reading means for reading an electrical signal characteristic of a heteropolymer molecule being sequenced from the pair of electrodes as the molecule being sequenced becomes electrically connected to the electrodes; (b) binding a DNA polymer to the DNA-binding protein; (c) binding a heteropolymer for sequencing to the DNA polymer; (d) arranging the bead to a first side of the constriction; and (e) sequencing the heteropolymer by reading the electrical signals thereof as the heteropolymer passes through the constriction.

In another aspect, the present disclosure relates to a method for regulating the speed of a heteropolymer passing through a constriction in a sequencing apparatus. The method can include: (a) attaching a DNA-binding protein to a bead, the bead including a size greater than a size of a constriction of a sequencing apparatus; (b) binding a DNA polymer to the DNA-binding protein; (c) binding a heteropolymer for sequencing by the sequencing apparatus to the DNA polymer; (d) arranging the bead to a first side of the constriction of the sequencing apparatus, wherein the first side of the constriction is in fluid communication with a reservoir having free nucleotides; and (e) regulating a speed of the heteropolymer for sequencing through the constriction by varying a concentration of the free nucleotides in the reservoir.

In some embodiments, the apparatus includes a recognition tunneling junction, such as those described below.

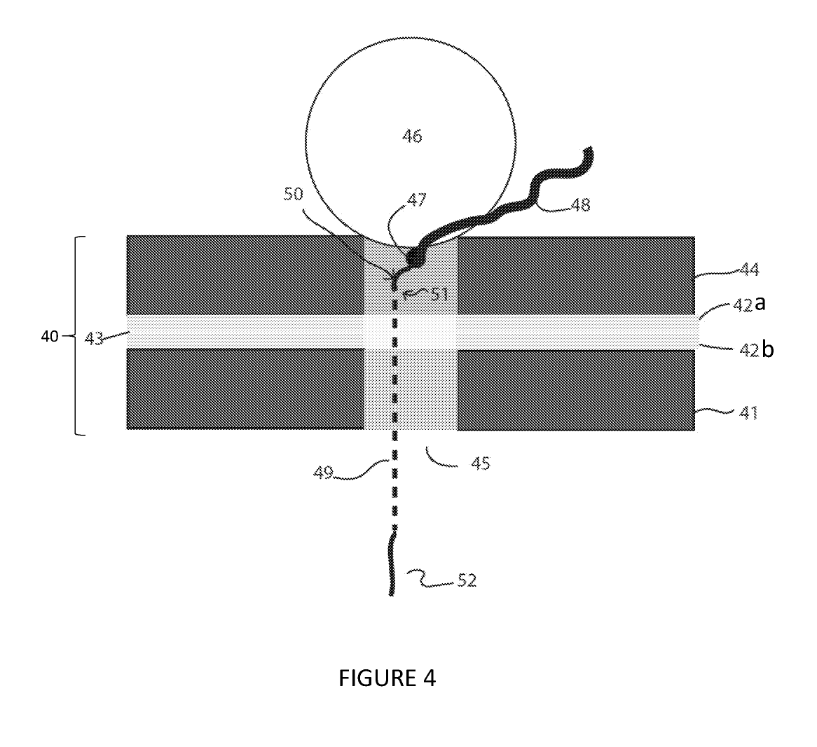

A general scheme of some of the embodiments is shown in FIG. 4. Here, the recognition tunneling junction includes layered substrate 40 which is comprised of a lower support membrane 41, a pair of metal electrodes 42a and 42b separated by a thin dielectric layer 43, a top dielectric layer 44, and a pore 45. The lower support membrane 41 is in contact with the metal electrode 42b. The top dielectric layer 44 is in contact with the metal electrode 42b. The metal electrodes 42a and 42b are sandwiched by the lower support membrane 41 and the top dielectric layer 44. The pore 45 extends continuously from a side of the lower support membrane 41 to a side of the top dielectric layer 44. The pore 45 can be drilled through the stack to expose the metal (42)—insulator (43)—metal (42) junction and the metal surface can be functionalized with recognition molecules (e.g., see U.S. Pat. No. 9,395,352). Non-limiting examples of recognition molecules can include mercaptobenzoic acid, 4-mercaptobenzcarbamide, imidazole-2-carboxide, and 4-carbamonylphenyldithiocarbamate.

The metal electrodes 42a and 42b can include palladium gold, platinum, or a combination thereof. The lower support membrane 41 can include a dielectric, such as silicon nitride, silicon dioxide, and other semiconductor or metal oxide. The lower support membrane 41 can be in contact with a first fluid reservoir. The top dielectric layer 44 can include a dielectric such as silicon nitride, silicon dioxide, and other semiconductor or metal oxide. The top dielectric layer 44 serves to isolate the top electrode 42a from a fluid (e.g., an aqueous electrolyte) in a second fluid reservoir. The fluid can serve as a transport medium for the molecules to be analyzed. The first and second fluid reservoirs can be in fluidic communication through the pore 45.

The lower support membrane 41 can have a thickness of about 5 nm to about 500 nm, about 10 nm to about 400 nm, about 20 nm to about 300 nm, about 20 nm to about 200 nm, or about 20 nm to about 100 nm. The metal electrodes 42a and 42b can each have a thickness of about 1 nm to about 20 nm, about 1 nm to about 15 nm, or about 1 nm to about 10 nm. The thin dielectric layer 43 can have a thickness of about 0.5 nm to about 10 nm, about 1 nm to about 5 nm, or about 1 nm to about 3 nm. The top dielectric layer 44 can have a thickness of about 5 nm to about 500 nm, about 10 nm to about 400 nm, about 20 nm to about 300 nm, about 20 nm to about 200 nm, or about 20 nm to about 100 nm. The pore 45 can have a diameter of about 2 to about 50 nm, about 5 nm to 40 nm, or about 5 nm to about 30 nm.

In some embodiments, a molecular motor (47) is attached to a bead 46 that is larger in size than the pore 45, thus attaching the motor 47 to the top electrode 42a via the top dielectric layer 44 once the bead 46 is pulled into the pore 45 by means of an attached charged molecule. In some embodiments, the bead 46 can be larger in diameter than the pore 45 by at least 1%, at least 2%, at least 3%, at least 4%, at least 5%, at least 6%, at least 7%, at least 8%, at least 9%, at least 10%, at least 15%, or at least 20%.

In some embodiments, a bead of somewhat smaller diameter can still be trapped at the opening of the device using a chemical approach. For example, if the opening in the top dielectric layer 44 in FIG. 4 is chemically modified to trap the bead, a bead carrying more than one streptavidin molecule can be trapped by treating the surface of the top coating 44 with a biotinylated silane.

In some embodiments, the molecular motor 47 may be a DNA polymerase attached to a double stranded DNA 48 at a double-single strand junction. The single stranded tail 50 that protrudes from the polymerase 47 is attached at its end 51 to the molecule to be sequenced (dashed line 49). In the event that the molecule to be sequenced is uncharged, it can also be ligated at its far end to a second piece of DNA 52 which will serve as a charged thread to pull the molecule 49 through the pore by means of electrophoresis. For example, the first and second fluid reservoirs can each include a reference electrode. By applying a voltage between these reference electrodes having a polarity opposite to that of the second piece of DNA 52, electrophoresis would pull the molecule 49 through the pore.

Examples of DNA polymerases include, but are not limited to, DNA polymerase I, DNA polymerase II, DNA polymerase III, DNA polymerase IV, DNA polymerase V, polymerase β, polymerase λ, polymerase σ, polymerase μ, polymerase α, polymerase δ, polymerase ε, polymerase η, polymerase ι, polymerase κ, polymerase Rev1, polymerase ζ, telomerase, polymerase γ, polymerase θ, polymerase ν, reverse transcriptase, polymerase T4, polymerase T7, and polymerase ϕ29 DNA.

DNA-binding proteins include transcription factors which modulate the process of transcription, various polymerases, nucleases which cleave DNA molecules, and histones which are involved in chromosome packaging and transcription in the cell nucleus. DNA-binding proteins can incorporate such domains as the zinc finger, the helix-turn-helix, and the leucine zipper (among many others) that facilitate binding to nucleic acid. There are also more unusual examples such as transcription activator like effectors. Examples of DNA-binding proteins include, but are not limited to, c-myb, AAF, abd-A, Abd-B, ABF-2, ABF1, ACE2, ACF, ADA2, ADA3, Adf-1, Adf-2a, ADR1, AEF-1, AF-2, AFP1, AGIE-BP1, AhR, AIC3, AIC4, AID2, AIIN3, ALF1B, alpha-1, alpha-CP1, alpha-CP2a, alpha-CP2b, alpha-factor, alpha-PAL, alpha2uNF1, alpha2uNF3, alphaA-CRYBP1, alphaH2-alphaH3, alphaMHCBF1, aMEF-2, AML1, AnCF, ANF, ANF-2, Antp, AP-1, AP-2, AP-3, AP-5, APETALA1, APETALA3, AR, ARG RI, ARG RII, Arnt, AS-C T3, AS321, ASF-1, ASH-1, ASH-3b, ASP, AT-13P2, ATBF1-A, ATF, ATF-1, ATF-3, ATF-3deltaZIP, ATF-adelta, ATF-like, Athb-1, Athb-2, Axial, abaA, ABF-1, Ac, ADA-NF1, ADD1, Adf-2b, AF-1, AG, AIC2, AIC5, ALF1A, alpha-CBF, alpha-CP2a, alpha-CP2b, alpha-IRP, alpha2uNF2, alphaH0, AmdR, AMT1, ANF-1, Ap, AP-3, AP-4, APETALA2, aRA, ARG RIII, ARP-1, Ase, ASH-3a, AT-BP1, ATBF1-B, ATF-2, ATF-a, ATF/CREB, Ato, B factor, B″, B-Myc, B-TFIID, band I factor, BAP, Bcd, BCFI, Bcl-3, beta-1, BETA1, BETA2, BF-1, BGP1, BmFTZ-F1, BP1, BR-C Z1, BR-C Z2, BR-C Z4, Brachyury, BRF1, BrlA, Brn-3a, Brn-4, Brn-5, BUF1, BUF2, B-Myb, BAF1, BAS1, BCFII, beta-factor, BETA3, BLyF, BP2, BR-C Z3, brahma, byr3, c-abl, c-Ets-1, c-Ets-2, c-Fos, c-Jun, c-Maf, c-myb, c-Myc, c-Qin, c-Rel, C/EBP, C/EBPalpha, C/EBPbeta, C/EBPdelta, C/EBPepsilon, C/EBPgamma, C1, CAC-binding protein, CACCC-binding factor, Cactus, Cad, CAD1, CAP, CArG box-binding protein, CAUP, CBF, CBP, CBTF, CCAAT-binding factor, CCBF, CCF, CCK-1a, CCK-1b, CD28RC, CDC10, Cdc68, CDF, cdk2, CDP, Cdx-1, Cdx-2, Cdx-3, CEBF, CEH-18, CeMyoD, CF1, Cf1a, CF2-I, CF2-II, CF2-III, CFF, CG-1, CHOP-10, Chox-2.7, CIIIB1, Clox, Cnc, CoMP1, core-binding factor, CoS, COUP, COUP-TF, CP1, CP1A, CP1B, CP2, CPBP, CPC1, CPE binding protein CPRF-1, CPRF-2, CPRF-3, CRE-BP1, CRE-BP2, CRE-BP3, CRE-BPa, CreA, CREB, CREB-2, CREBomega, CREMalpha, CREMbeta, CREMdelta, CREMepsilon, CREMgamma, CREMtaualpha, CRF, CSBP-1, CTCF, CTF, CUP2, Cut, Cux, Cx, cyclin A, CYS3, D-MEF2, Da, DAL82, DAP, DAT1, DBF-A, DBF4, DBP, DBSF, dCREB, dDP, dE2F, DEF, Delilah, delta factor, deltaCREB, deltaE1, deltaEF1, deltaMax, DENF, DEP, DF-1, Dfd, dFRA, dioxin receptor, dJRA, D1, DII, D1x, DM-SSRP1, DMLP1, DP-1, Dpn, Dr1, DRTF, DSC1, DSP1, DSXF, DSXM, DTF, E, E1A, E2, E2BP, E2F, E2F-BF, E2F-I, E4, E47, E4BP4, E4F, E4TF2, E7, E74, E75, EBF, EBF1, EBNA, EBP, EBP40, EC, ECF, ECH, EcR, eE-TF, EF-1A, EF-C, EF1, EFgamma, Egr, eH-TF, EIIa, EivF, EKLF, Elf-1, Elg, Elk-1, ELP, Elt-2, EmBP-1, embryo DNA binding protein, Emc, EMF, Ems, Emx, En, ENH-binding protein, ENKTF-1, epsilonF1, ER, Erg, Esc, ETF, Eve, Evi, Evx, Exd, Ey, f(alpha-epsilon), F-ACT1, f-EBP, F2F, factor 1-3, factor B1, factor B2, factor delta, factor I, FBF-A1, Fbf1, FKBP59, Fkh, F1bD, F1h, Fli-1, FLV-1, Fos-B, Fra-2, FraI, FRG Y1, FRG Y2, FTS, Ftz, Ftz-F1, G factor, G6 factor, GA-BF, GABP, GADD 153, GAF, GAGA factor, GAL4, GAL80, gamma-factor, gammaCAAT, gammaCAC, gammaOBP, GATA-1, GATA-2, GATA-3, GBF, GC1, GCF, GCF, GCN4, GCR1, GE1, GEBF-I, GF1, GFI, Gfi-1, GFII, GHF-5, GL1, Glass, GLO, GM-PBP-1, GP, GR, GRF-1, Gsb, Gsbn, Gsc, Gt, GT-1, Gtx, H, H16, H1lTF1, H2Babp1, H2RIIBP, H2TF1, H4TF-1, HAC1, HAP1, Hb, HBLF, HBP-1, HCM1, heat-induced factor, HEB, HEF-1B, HEF-1T, HEF-4C, HEN1, HES-1, HIF-1, HiNF-A, HIP1, HIV-EP2, Hlf, HMBI, HNF-1, HNF-3, Hox11, HOXA1, HOXA10, HOXA10PL2, HOXA11, HOXA2, HOXA3, HOXA4, HOXA5, HOXA7, HOXA9, HOXB1, HOXB3, HOXB4, HOXB5, HOXB6, HOXB7, HOXB8, HOXB9, HOXC5, HOXC6, HOXC8, HOXD1, HOXD10, HOXD11, HOXD12, HOXD13, HOXD4, HOXD8, HOXD9, HP1 site factor, Hp55, Hp65, HrpF, HSE-binding protein, HSF1, HSF2, HSF24, HSF3, HSF30, HSF8, hsp56, Hsp90, HST, HSTF, I-POU, IBF, IBP-1, ICER, ICP4, ICSBP, Id1, Id2, Id3, Id4, IE1, EBP1, IEFga, IF1, IF2, IFNEX, IgPE-1, IK-1, IkappaB, Il-1 RF, IL-6 RE-BP, 1L-6 RF, ILF, ILRF-A, IME1, INO2, INSAF, IPF1, IRBP, IRE-ABP, IREBF-1, IRF-1, ISGF-1, Isl-1, ISRF, ITF, IUF-1, Ixr1, JRF, Jun-D, JunB, JunD, K-2, kappay factor, kBF-A, KBF1, KBF2, KBP-1, KER-1, Ker1, KN1, Kni, Knox3, Kr, kreisler, KRF-1, Krox-20, Krox-24, Ku autoantigen, KUP, Lab, LAC9, LBP, Lc, LCR-F1, LEF-1, LEF-1S, LEU3, LF-A1, LF-B1, LF-C, LF-H3beta, LH-2, Lim-1, Lim-3, lin-11, lin-31, lin-32, LIP, LIT-1, LKLF, Lmx-1, LRF-1, LSF, LSIRF-2, LVa, LVb-binding factor, LVc, LyF-1, Lyl-1, M factor, M-Twist, M1, m3, Mab-18, MAC1, Mad, MAF, MafB, MafF, MafG, MafK, Ma163, MAPF1, MAPF2, MASH-1, MASH-2, mat-Mc, mat-Pc, MATa1, MATalpha1, MATalpha2, MATH-1, MATH-2, Max1, MAZ, MBF-1, MBP-1, MBP-2, MCBF, MCM1, MDBP, MEB-1, Mec-3, MECA, mediating factor, MEF-2, MEF-2C, MEF-2D, MEF1, MEP-1, Meso1, MF3, Mi, MIF, MIG1, MLP, MNB1a, MNF1, MOK-2, MP4, MPBF, MR, MRF4, MSN2, MSN4, Msx-1, Msx-2, MTF-1, mtTF1, muEBP-B, muEBP-C2, MUF1, MUF2, Mxi1, Myef-2, Myf-3, Myf-4, Myf-5, Myf-6, Myn, MyoD, myogenin, MZF-1, N-Myc, N-Oct-2, N-Oct-3, N-Oct-4, N-Oct-5, Nau, NBF, NC1, NeP1, Net, NeuroD, neurogenin, NF III-a, NF-1, NF-4FA, NF-AT, NF-BA1, NF-CLE0a, NF-D, NF-E, NF-E1b, NF-E2, NF-EM5, NF-GMa, NF-H1, NF-IL-2A, NF-InsE1, NF-kappaB, NF-lambda2, NF-MHCIIA, NF-muE1, NF-muNR, NF-S, NF-TNF, NF-U1, NF-W1, NF-X, NF-Y, NF-Zc, NFalpha1, NFAT-1, NFbetaA, NFdeltaE3A, NFdeltaE4A, NFe, NFE-6, NFH3-1, NFH3-2, NFH3-3, NFH3-4, NGFI-B, NGFI-C, NHP, Nil-2-a, NIP, NIT2, Nkx-2.5, NLS1, NMH7, NP-III, NP-IV, NP-TCII, NP-Va, NRDI, NRF-1, NRF-2, Nrf1, Nrf2, NRL, NRSF form 1, NTF, NUC-1, Nur77, OBF, OBP, OCA-B, OCSTF, Oct-1, Oct-10, Oct-11, Oct-2, Oct-2.1, Oct-2.3, Oct-4, Oct-5, Oct-6, Oct-7, Oct-8, Oct-9, Oct-B2, Oct-R, Octa-factor, octamer-binding factor, Odd, Olf-1, Opaque-2, Otd, Otx1, Otx2, Ovo, P, P1, p107, p130, p28 modulator, p300, p38erg, p40x, p45, p49erg, p53, p55, p55erg, p58, p65de1ta, p67, PAB1, PacC, Pap1, Paraxis, Pax-1, Pax-2, Pax-3, Pax-5, Pax-6, Pax-7, Pax-8, Pb, Pbx-1a, Pbx-1b, PC, PC2, PC4, PC5, Pcr1, PCRE1, PCT1, PDM-1, PDM-2, PEA1, PEB1, PEBP2, PEBP5, Pep-1, PF1, PGA4, PHD1, PHO2, PHO4, PHO80, Phox-2, Pit-1, PO-B, pointedP1, Pou2, PPAR, PPUR, PPYR, PR, PR A, Prd, PrDI-BF1, PREB, Prh protein a, protein b, protein c, protein d, PRP, PSE1, PTF, Pu box binding factor, PU.1, PUB1, PuF, PUF-I, Pur factor, PUT3, pX, qa-1F, QBP, R, R1, R2, RAd-1, RAF, RAP1, RAR, Rb, RBP-Jkappa, RBP60, RC1, RC2, REB1, Re1A, Re1B, repressor of CAR1 expression, REX-1, RF-Y, RF1, RFX, RGM1, RIM1, RLM1, RME1, Ro, RORalpha, Rox1, RPF1, RPGalpha, RREB-1, RRF1, RSRFC4, runt, RVF, RXR-alpha, RXR-beta, RXR-beta2, RXR-gamma, S-CREM, S-CREMbeta, S8, SAP-1a, SAP1, SBF, Sc, SCBPalpha, SCD1/BP, SCM-inducible factor, Scr, Sd, Sdc-1, SEF-1, SF-1, SF-2, SF-3, SF-A, SGC1, SGF-1, SGF-2, SGF-3, SGF-4, SIF, SIII, Sim, SIN1, Skn-1, SKO1, Slp1, Sn, SNP1, SNF5, SNAPC43, Sox-18, Sox-2, Sox-4, Sox-5, Sox-9, Sox-LZ, Sp1, spE2F, Sph factor, Spi-B, Sprm-1, SRB10, SREBP, SRF, SRY, SSDBP-1, ssDBP-2, SSRP1, STAF-50, STAT, STAT1, STAT2, STAT3, STAT4, STATS, STATE, STC, STD1, Ste11, Ste12, Ste4, STM, Su(f), SUM-1, SWI1, SWI4, SWI5, SWI6, SWP, T-Ag, t-Pou2, T3R, TAB, all TAFs including subunits, Tal-1, TAR factor, tat, Tax, TBF1, TBP, TCF, TDEF, TEA1, TEC1, TEF, tel, Tf-LF1, TFE3, all TFII related proteins, TBA1a, TGGCA-binding protein, TGT3, Th1, TIF1, TIN-1, TIP, T11, TMF, TR2, Tra-1, TRAP, TREB-1, TREB-2, TREB-3, TREF1, TREF2, Tsh, TTF-1, TTF-2, Ttk69k, TTP, Ttx, TUBF, Twi, TxREBP, TyBF, UBP-1, Ubx, UCRB, UCRF-L, UF1-H3beta, UFA, UFB, UHF-1, UME6, Unc-86, URF, URSF, URTF, USF, USF2, v-ErbA, v-Ets, v-Fos, v-Jun, v-Maf, v-Myb, v-Myc, v-Qin, v-Rel, Vab-3, vaccinia virus DNA-binding protein, Vav, VBP, VDR, VETF, vHNF-1, VITF, Vmw65, Vp1, Vp16, Whn, WT1, X-box binding protein, X-Twist, X2BP, XBP-1, XBP-2, XBP-3, XF1, XF2, XFD-1, XFD-3, xMEF-2, XPF-1, XrpFI, W, XX, yan, YB-1, YEB3, YEBP, Yi, YPF1, YY1, ZAP, ZEM1, ZEM2/3, Zen-1, Zen-2, Zeste, ZF1, ZF2, Zfh-1, Zfh-2, Zfp-35, ZID, Zmhoxla, and Zta.

In some embodiments, the DNA-binding protein is a helicase. In some embodiments, the DNA-binding protein is an endonuclease. In some embodiments, the DNA-binding protein is a DNA repair protein.

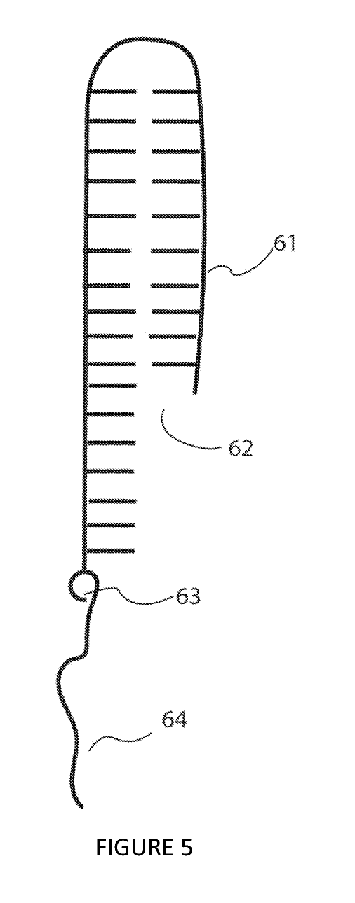

In some embodiments, referring to FIG. 5, the molecule to be sequenced 64 may be first tethered to a DNA oligomer 61 by means of a suitable linker 63 (see below). The DNA oligomer is designed to form a hairpin with a double strand-single strand junction that serves as a priming site for the DNA polymerase to bind. Examples of suitable linkers include, but are not limited to, polyethyleneglycol and other water-soluble, flexible polymers including sugars (e.g., chitin or chitosan). In some embodiments, the suitable linker 63 can be polyethyleneglycol.

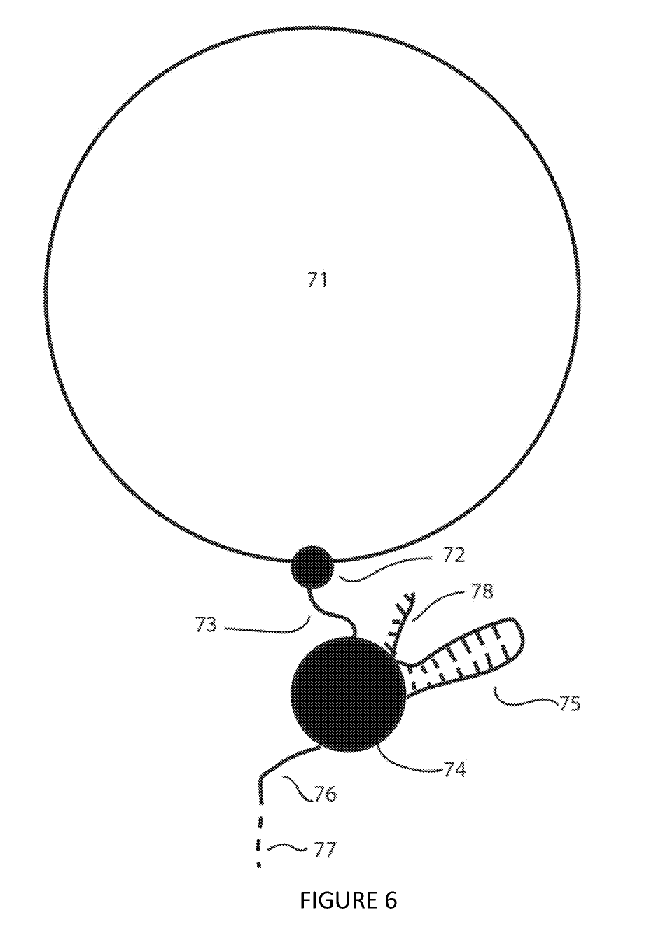

In some embodiments, referring to FIG. 6, the DNA polymerase 74 (such as a ϕ29) may be attached to bead 71 by means of a biotinylated 73 residue that attaches to a streptavidin 72 molecule on the surface of the bead. As the molecule to be sequenced 77 is pulled into the pore by the electrophoretic force, the single-stranded DNA tail 76 is also pulled, so that the hairpin 75 is unwound, producing the single strand 78 as well as a substantial resistance force which will produce the desired slowing of the electrophoretic translocation of the molecule 77.

One of skill in the art will appreciate that incorporating an abasic strand into the construct (as shown in FIG. 1) may allow this process to be carried out in the presence of nucleotides. When the strand with the abasic section is pulled off, DNA synthesis begins (in the presence of free nucleotides) so that the molecule to be sequenced may be pulled up again as the hairpin 75 became elongated again, thus resequencing the target molecule 77 at a speed controlled by the concentration of free nucleotides. A higher concentration of the free nucleotides results in faster movement of the molecule to be sequenced in the constriction.

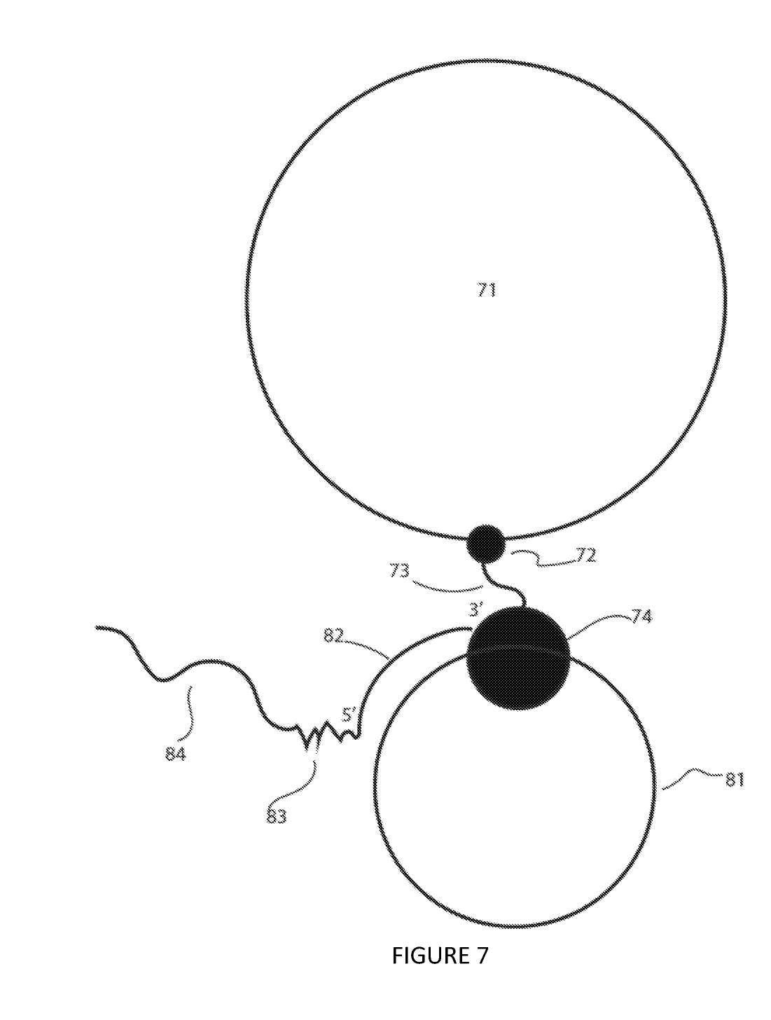

In some embodiments, rolling circle amplification (RCA) may be exploited. Referring to FIG. 7, a polymerase 74 may be bound to a bead 71 by means of a biotinylated tether 73 attached to a streptavidin 72 on the bead. In the present embodiments, the polymerase 74 may be incubated with a solution of a circular sequence of single stranded DNA 81 hybridized to a primer sequence 82 such that the polymerase binds at the 3′ end of the primer. The primer is modified at its 5′ end with a short flexible tether 83 (such as polyethyleneglycol).

The molecule to be sequenced 84 may be attached to the tether by a covalent linkage of the kind described below. In the presence of nucleotides, the double stranded region is extended until the polymerase reaches the 5′ end of the primer. At this point, the polymerase can push the synthesized strand off the circle at a rate that depends on the concentration of free nucleotides, continuing the amplification. This can allow the molecule to be sequenced 84 to be pulled down into the reading junction where its sequence can be read. Once again, the molecule to be sequenced can be attached to a nucleic acid ‘thread molecule’ if its charge is insufficient, as shown in FIG. 4.

Some of the embodiments have been described in the context of a layered tunnel junction with a pore running through the layers. However, the same principles can apply to a tunnel junction in which the electrodes lay opposite on another in a plane, separated by a small gap that forms a tunnel junction. In this case, the constriction that can be used to transport the molecules to the junction would be a narrow channel lying across the junction. The mouth of the constriction would then serve as a point to trap the bead (46) so that the motion of the polymer down the channel could be controlled as described above.

A component of some of the embodiments includes a method for tethering the molecule to be sequenced to the 5′ or 3′ end of DNA. We have described a method whereby peptide chains can be reliably attached to DNA at their N-terminus (Biswas, Song et al. 2015), thus allowing peptides to be sequenced via the characteristic signals produced by their amino acid residues (Zhao, Ashcroft et al. 2014) if they are pulled through the tunnel junction in the manner outlined in some of the embodiments of the present disclosure. The contents of these references are incorporated by reference in their entireties.

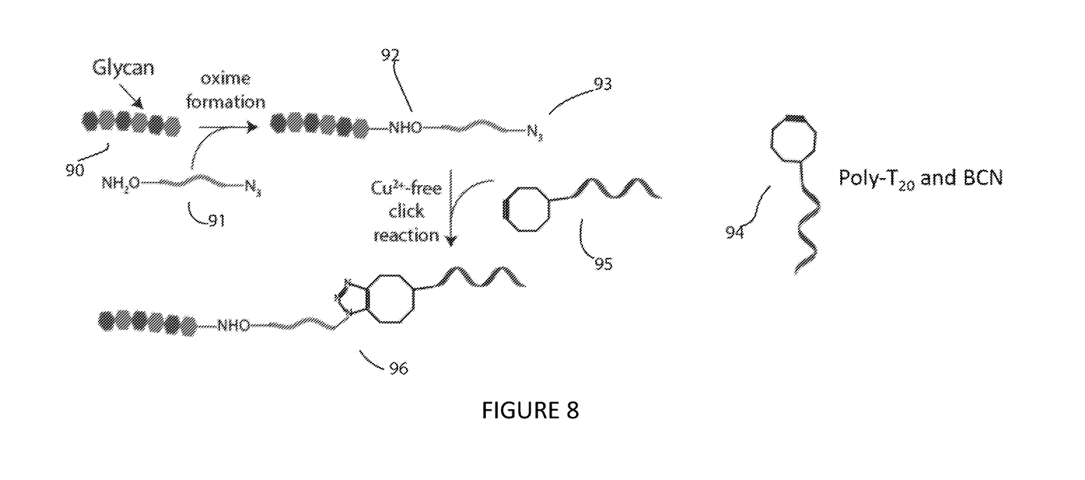

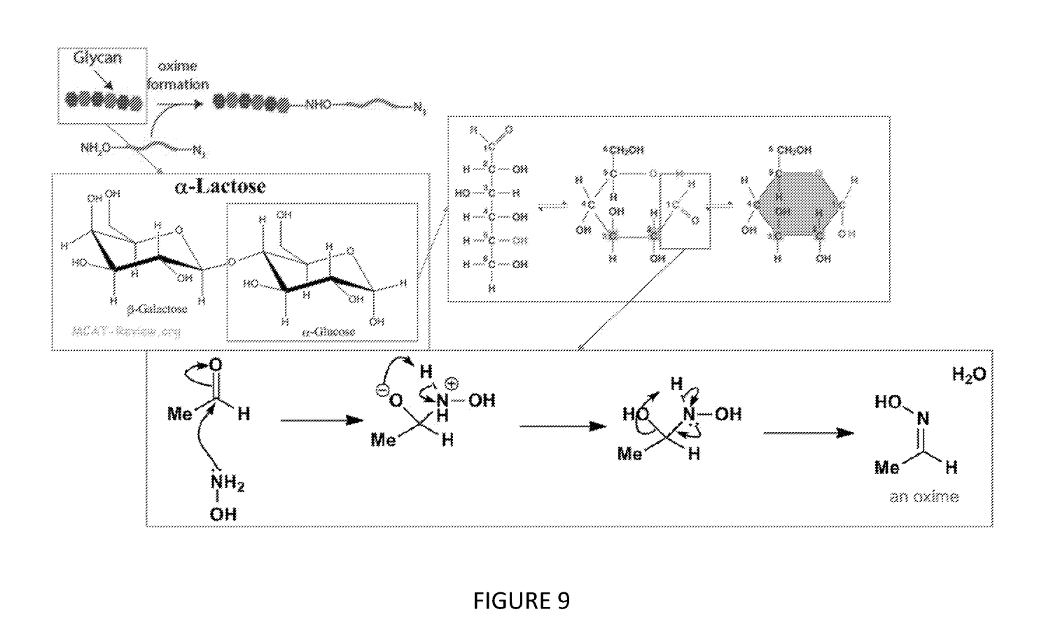

In the present disclosure, we also describe a method for attaching oligosaccharides to a DNA molecule. Referring to FIG. 8, a scheme is illustrated for attaching an azide to the reducing end of a glycan. A flexible linker (e.g., polyethyleneglycol, [PEG]₆) terminated at one end with an aminooxy group, and at the other end with an azide—N₃-[PEG₅]-CH₂CH₂ONH₂ (91 on FIG. 8) is used. The flexible linker is reacted with the lactose-terminated glycan 90 for about 8 hours in 100 mM acetate buffer (pH 4.1). A nearly 100% yield of oxime coupled 92 glycan terminated in an azide 93 is obtained (this reaction is further illustrated in FIG. 9). A symmetrical cyclooctyne (BCN: bicyclo[6.1.0]nonyne), 94) attached to a DNA will reliably couple the DNA conjugate via copper-free click chemistry 95 to form the desired product 96. In FIG. 8, reactions are shown for the coupling of a T₂₀ oligomer, but it will be recognized that coupling of any of the nucleic acid constructs in the forgoing disclosure can follow the same path.

Any and all references to publications or other documents, including but not limited to, patents, patent applications, articles, webpages, books, etc., presented in the present application, are herein incorporated by reference in their entirety.

Example embodiments of the devices, systems and methods have been described herein. These embodiments have been described for illustrative purposes only and are not limiting. Other embodiments are possible and are covered by the disclosure, which will be apparent from the teachings contained herein. Thus, the breadth and scope of the disclosure should not be limited by any of the above-described embodiments, but should be defined only in accordance with claims supported by the present disclosure and their equivalents. Moreover, embodiments of the subject disclosure may include methods, systems and devices that include any and all elements from any other disclosed methods, systems, and devices, including any and all elements corresponding to sequencing molecules and the preparation of such molecules for sequencing. In other words, elements from one or another disclosed embodiments may be interchangeable with elements from other disclosed embodiments. In addition, one or more features/elements of disclosed embodiments may be removed and still result in patentable subject matter (and thus, resulting in yet more embodiments of the subject disclosure). Correspondingly, some embodiments of the present disclosure may be patentably distinct from one and/or another reference by specifically lacking one or more elements/features. In other words, claims to certain embodiments may contain negative limitation to specifically exclude one or more elements/features resulting in embodiments which are patentably distinct from the prior art which include such features/elements.

### CITATIONS

Apweiler, R., et al. (1999). “On the frequency of protein glycosylation, as deduced from analysis of the SWISS-PROT database1.” Biochimica et Biophysica Acta 1473: 4-8.
Biswas, S., et al. (2015). “Click Addition of a DNA Thread to the N-Termini of Peptides for Their Translocation through Solid-State Nanopores.” ACS Nano 9 (10): 9652-9664.

### Fennouri, A., et al. (2012). “Single Molecule Detection of Glycosaminoglycan Hyaluronic Acid Oligosaccharides and Depolymerization Enzyme Activity Using a Protein Nanopore.” ACS Nano 6 (11): 9672-9678.

Hart, G. W. and R. J. Copeland (2010). “Glycomics hits the big time.” Cell 143 (5): 672-676.
Hofmann, J., et al. (2015). “Identification of carbohydrate anomers using ion mobility-mass spectrometry.” Nature 526 (7572): 241-244.
Kawai, T. and S. Akira (2009). “The roles of TLRs, RLRs and NLRs in pathogen recognition.” International Immunology 21 (4): 317-337.
Manrao, E. A., et al. (2012). “Reading DNA at single-nucleotide resolution with a mutant MspA nanopore and phi29 DNA polymerase.” Nat Biotechnol 30 (4): 349-353.
Nagy, G. and N. L. Pohl (2015). “Monosaccharide identification as a first step toward de novo carbohydrate sequencing: mass spectrometry strategy for the identification and differentiation of diastereomeric and enantiomeric pentose isomers.” Analytical Chemistry 87 (8): 4566-4571.
Ohtsubo, K. and J. D. Marth (2006). “Glycosylation in cellular mechanisms of health and disease.” Cell 126 (5): 855-867.
Parodi, A. J. (2000). “Protein glucosylation and its role in protein folding.” Annu Rev Biochem 69: 69-93.
Pinho, S. S. and C. A. Reis (2015). “Glycosylation in cancer: mechanisms and clinical implications.” Nature Reviews: Cancer 15 (9): 540-555.

### Werz, D. B., et al. (2007). “Exploring the Structural Diversity of Mammalian Carbohydrates (“Glycospace”) by Statistical Databank Analysis.” ACS Chemical Biology 2 (10): 685-691.

Zhang, X. L. (2006). “Roles of glycans and glycopeptides in immune system and immune-related diseases.” Curr Med Chem 13 (10): 1141-1147.
Zhao, Y., et al. (2014). “Single-molecule spectroscopy of amino acids and peptides by recognition tunnelling.” Nature Nanotechnology 9: 466-473.
Zhao, Y. Y., et al. (2008). “Functional roles of N-glycans in cell signaling and cell adhesion in cancer.” Cancer Science 99 (7): 1304-1310.

1. An apparatus for sequencing a heteropolymer comprising:
a substrate;
a pair of electrodes proximate to or within the constriction and separated by a gap of between 0.5 to 10 nm;
a constriction arranged within the substrate and configured with a size and operatively arranged with the gap such that a heteropolymer molecule to be sequenced passes through the constriction;
means for reading an electrical signal characteristic of the molecule from the pair of electrodes as the heteropolymer molecule passes through the constriction and becomes electrically connected with the electrodes;
a bead having a size that is greater than a size of the constriction;
a DNA-binding protein attached to the bead; and
a DNA polymer bound to the DNA-binding protein and configured to bind with a heteropolymer for sequencing by the apparatus.

2. The apparatus of claim 1, wherein the heteropolymer is not a nucleic acid.

3. The apparatus of claim 1, wherein the size of the bead is such that it cannot move through the constriction.

4. The apparatus of claim 1, wherein the size of the bead is such that it cannot move into the constriction.

5. The apparatus of claim 1, wherein the heteropolymer is selected from the group consisting of an oligosaccharide, a polysaccharide, a peptide, a protein and a glycoprotein.

6. (canceled)

7. (canceled)

8. (canceled)

9. The apparatus of claim 1, wherein the heteropolymer for sequencing is tethered to a charged polymer.

10. The apparatus of claim 9, wherein the tethering of the charged polymer is configured to be drawn into the constriction.

11. The apparatus of claim 1, wherein the DNA-binding protein comprises a DNA polymerase.

12. The apparatus of claim 1, wherein the constriction has a diameter of between 5 to 40 nm.

13. (canceled)

14. (canceled)

15. (canceled)

16. (canceled)

17. A method for sequencing a heteropolymer in a sequencing apparatus having a constriction, the method comprising:
attaching a DNA-binding protein to a bead, the bead including a size greater than a size of a constriction of a sequencing apparatus, the sequencing apparatus further including a substrate, the constriction arranged within the substrate and configured with a size and operatively arranged with a pair of electrodes separated by a gap of between 0.5 to 10 nm such that a heteropolymer molecule to be sequenced passes through the constriction, reading means for reading an electrical signal characteristic of a heteropolymer molecule being sequenced from the pair of electrodes as the molecule being sequenced becomes electrically connected to the electrodes;
binding a DNA polymer to the DNA-binding protein;
binding a heteropolymer for sequencing to the DNA polymer;
arranging the bead to a first side of the constriction; and
sequencing the heteropolymer by reading the electrical signals thereof as the heteropolymer passes through the constriction.

18. The method of claim 17, wherein the heteropolymer is not a nucleic acid.

19. The method of claim 17, wherein the heteropolymer is selected from the group consisting of an oligosaccharide, a polysaccharide, a peptide, a protein, and a glycoprotein.

20. The method of claim 17, wherein the DNA-binding protein comprises a DNA polymerase.

21. A method for regulating a speed of a heteropolymer for sequencing as the heteropolymer passes through a constriction of a sequencing apparatus, the method comprising:
attaching a DNA-binding protein to a bead, the bead including a size greater than a size of a constriction of a sequencing apparatus;
binding a DNA polymer to the DNA-binding protein;
binding a heteropolymer for sequencing by the sequencing apparatus to the DNA polymer;
arranging the bead to a first side of the constriction of the sequencing apparatus, wherein the first side of the constriction is in fluid communication with a reservoir having free nucleotides; and
regulating a speed of the heteropolymer for sequencing through the constriction by varying a concentration of the free nucleotides in the reservoir.

22. The method of claim 21, wherein the concentration of the free nucleotides is increased such that the heteropolymer for sequencing increases speed through the constriction.

23. The method of claim 21, wherein the heteropolymer is not a nucleic acid.

24. The method of claim 21, wherein the heteropolymer is selected from the group consisting of an oligosaccharide, a polysaccharide, a peptide, a protein, and a glycoprotein.

25. The method of claim 21, wherein the DNA-binding protein comprises a DNA polymerase.

26. The method of claim 21, wherein the DNA polymer includes an abasic section.
